(19) United States
(12) Patent Application Publication
(10) Pub. No.: US 2025/0177264 A1
(43) Pub. Date: Jun. 5, 2025

(54) COMBINATION OF LINOLAL WITH FRUITY AND/OR SPICY FRAGRANCES

(71) Applicant: Symrise AG, Holzminden (DE)

(72) Inventors: Bernd HÖLSCHER, Halle (DE); Lara-Joy KLEINE-BENNE, Holzminden (DE); Theo Belmas, Holzminden (DE)

(21) Appl. No.: 18/845,252

(22) PCT Filed: Mar. 9, 2022
(86) PCT No.: PCT/EP2022/056069

§ 371 Date: Sep. 9, 2024

(51) Int. Cl. A61K 8/33; A61Q 13/00

(52) U.S. Cl. CPC A61K 8/33; A61Q 13/00

(57) ABSTRACT

Suggested is the use of linolal in small quantities as enhancer and/or modifier within fragrance compositions to positively affect fruity and spicy notes.

### FIELD OF THE INVENTION

The present invention relates to the use of linolal as enhancer, modifier and/or contributor of fragrances. Furthermore, the present invention relates to fragrance compositions, their use, and a method for imparting modifying and/or enhancing certain fragrance notes.

### BACKGROUND OF THE INVENTION

The perfume industry requires the constant development of novel fragrances to enhance the pallet of scents and satisfy consumer demands. However, the creation of a new fragrance or fragrance compositions involves a number of challenges. For example, it is necessary to select a specific composition of a few fragrances from an almost unlimited number of possible structures known from the prior art, in order to satisfy a specific need of the market and provide a product that matches the profile required by customers.

The combination of multiple known fragrances can guide the creation of scents that are perceived by the consumer as novel and exciting. In this process, it is necessary to combine several compounds for a single product. However, the cost of basic ingredient for fragrances or fragrance compositions is high, which results in an interest of the perfume industry to utilize fragrances as efficient as possible. The addition of further compounds to already known fragrances to create novel scents is an effective method to enhance the pallet of scents. Enhancing or modifying compounds boosting novel or known fragrances or compositions are in high demand as the resulting product can have superior properties regarding the overall perception of the scent. However, the identification of chemical compounds or compositions modifying and/or boosting the scent profile of known fragrances or fragrance compositions in order to create new scents is challenging. It is currently impossible to match compounds solely based on chemical structure to create a pleasant scent. Therefore, rigorous testing of fragrance combinations and concentrations by industry specialists is required to identify new candidates. In particular, the identification of additional compounds that are able to modify known fragrances by providing trace amounts of the additional compounds is highly desirable due to the possibility of creating a high number of novel scents from previously known fragrances or fragrance compositions by addition of said compounds. This method is cost effective due to the small amount necessary to enhance and/or modify the already known scent.

Thus, the underlying objective of the present intention was to provide a compound, which enables the positive modification of known scents and/or enhance the fragrance profile even in small quantities to increase the pallet of scents offered to costumers.

### DESCRIPTION OF THE INVENTION

This is solved by the use of linolal in combination with at least one other fragrance in order to impart, modify and/or enhance the scent profile of fragrance compositions.

Linolal is a mixture of C13 and C15 aldehydes.

In a preferred embodiment, the C13 and C15 aldehydes are present in the weight ratio of about 1:100 to about 100:1.

In a preferred embodiment, linolal is a mixture of compounds according to CAS Nr. 93821-14-8.

In a preferred embodiment, linolal is the hydroformylation products of alkenes C12 to C14.

In a preferred embodiment, linolal is the reaction mass of pentadecanal, tridecanal, 2-methyldodecanal and 2-methyltetradecanal.

Linolal is known by the fragrance industry and its fragrance profile is described as comprising notes related to aldehydes, fat, conifers and flowers.

Surprisingly, it has been found that the addition of small amounts of linolal to fragrance compositions greatly modified the scent profile of said composition in a positive way. In particular, spicy and/or fruity notes of fragrance compositions have been affected by the addition of linolal. The addition of small amounts of linolal has been shown to modify already present fruity notes in fragrance compositions, particularly imparting the sense of notes such as related to watermelon, raspberry, banana, pear, apricot, peach, mandarin peel, and citrus. Similarly, spicy notes were positively affected by addition of linolal, particularly imparting the sense of notes such as almond, coriander, basil, bergamot peel, eucalyptus, pepper, cinnamon, cassia, bay oil, caraway, anise, and cardamom. These results, however, are particularly surprising, since linolal does not have any fruity or spicy notes by itself and has not been described to influence the scent profile of fragrance compositions promoting said notes.

In connection with the preferred use for imparting, modifying and/or enhancing a fragrance note, it is also recognized that linolal excellently functions as a so-called booster (amplifier; enhancer).

More surprisingly, small amounts of linolal were able to enhance already existing spicy and/or fruity notes of fragrance compositions.

In connection with the preferred use according to the present invention, linolal is used in a sensory effective amount. A sensory effective amount is the total amount necessary for imparting a sensory effect alone or in combination with further compounds or compositions (=working amount).

In a preferred embodiment, linolal is used together with at least one fragrance and/or composition to create a fragrance composition. Said fragrance compositions are obtainable by methods known in the art such as simple mixing or homogenization of ingredients. The at least one other fragrance and/or composition can be any other fragrance or composition.

Therefore, the present invention is related to the use of linolal and at least one further compound and/or composition as fragrance composition, wherein linolal is contained in a sensory effective amount sufficient to impart, modify and/or enhance fruity and/or spicy notes of an existing fragrance.

In a preferred embodiment of the present invention fruity notes are selected from the group consisting of watermelon, raspberry, banana, pear, apricot, peach, mandarin peel, and citrus.

In a preferred embodiment of the present invention spicy notes are selected from the group consisting of almond, coriander, basil, bergamot peel, eucalyptus, pepper, cinnamon, cassia, bay oil, caraway, anise, and cardamom.

Linolal can be used in a variety of products and can be combined particularly advantageously with other fragrances in different proportions to form fragrance mixtures, and novel and original perfume compositions can also be created.

Accordingly, one aspect of the invention also relates to fragrance mixture and possibly further constituents (solvents or the like), which contain linolal.

In a preferred embodiment of the present invention the amount of linolal is in the range of 0.00001 to 99.9% by weight, based on the total weight of the fragrance composition, more preferably in the range of 0.001 to 5% by weight, and most preferably in the range of 0.05 to 1% by weight.

In a preferred embodiment of the present invention the weight ratio of linolal to the total amount of other fragrances is in the range of 1:100000 to 1:20, more preferably in the range of 1:20000 to 1:1000.

Examples of fragrances which may advantageously be combined with linolal in the context of the present invention can be found, for example, in S. Arctander, Perfume and Flavor Materials, Vol. I and II, Montclair, N. J. 1969, self-published, or K. Bauer et al., Common Fragrance and Flavor Materials, 4th Edition, Wiley-VCH, Weinheim 2001.

Specifically mentioned are: Extracts from natural raw materials such as essential oils, concretes, absolutes, resins, resinoids, balsams, tinctures such as: Ambergris tincture; Amyris oil; Angelica seed oil; Angelica root oil; Anise oil; Valerian oil; Basil oil; tree moss absolute; bay oil; mugwort oil; benzoeresin; bergamot oil; Beeswax absolue; Birch tar oil; Bitter almond oil; Savory oil; Bucco leaf oil; Cabreuva oil; Cade oil; Calmus oil; Camphor oil; Cananga oil; Cardamom oil; Cascarilla oil; Cassia oil; Cassie-absolue; Castoreum-absolue; Cedar leaf oil; Cedarwood oil; Cistus oil; Citronella oil; Citron oil; Copaiva balsam; Copaiva balsam oil; Coriander oil; Costus root oil; Cumin oil; Cypress oil; Davana oil; Dill herb oil; Dill seed oil; Eau de brouts-Absolue; Oak moss absolute; Elemi oil; Tarragon oil; Eucalyptus citriodora oil; Eucalyptus oil; Fennel oil; Spruce needle oil; Galbanum oil; Galbanum resin; Geranium oil; Grapefruit oil; Guaiac wood oil; Gurjun balsam; Gurjun balsam oil; Helichrysum absolute; Helichrysum oil; Ginger oil; Iris root absolute; Iris root oil; Jasmine absolute; Calamus oil; Chamomile oil blue; Chamomile oil roman; Carrot seed oil; Cascarilla oil; Pine needle oil; Spearmint oil; Caraway oil; labdanum oil; labdanum absolute; labdanum resin; lavandin absolute; Lavandin oil; Lavender absolute; Lavender oil; Lemongrass oil; Lovage oil; Lime oil distilled; Lime oil pressed; Linaloe oil; Litsea cubeba oil; Bay leaf oil; Mace oil; Marjoram oil; Mandarin oil; Massoir bark oil; Mimosa absolute; Musk grain oil; Musk tincture; Muscat sage oil; Nutmeg oil; Myrrh absolute; Myrrh oil; Myrtle oil; Clove leaf oil; Clove flower oil; Neroli oil; Olibanum absolute; Olibanum oil; Opopanax oil; orange blossom absolute; orange oil; origanum oil; palmarosa oil; patchouli oil; Perilla oil; Perubalsam oil; Parsley leaf oil; Parsley seed oil; Petitgrain oil; Peppermint oil; Pepper oil; Allspice oil; Pine oil; Poley oil; Rose absolute; Rosewood oil; Rose oil; Rosemary oil; Sage oil Dalmatian; Sage oil Spanish; Sandalwood oil; Celery seed oil; Spicy lavender oil; Star anise oil; Styrax oil; Tagetes oil; Fir needle oil; Tea tree oil; Turpentine oil; Thyme oil; Tolu balsam; Tonka absolue; tuberose-absolue; vanilla extract; violet leaf-absolue; verbena oil; vetiver oil; juniper berry oil; wine yeast oil; wormwood oil; wintergreen oil; ylang oil; hyssop oil; civet-absolue; cinnamon leaf oil; cinnamon bark oil and fractions thereof, or Ingredients isolated therefrom;

Individual odorants from the group of hydrocarbons, such as 3-carene; α-pinene; ß-pinene; a-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;

of the aliphatic alcohols such as Hexanol; octanol; 3-octanol; 2,6-dimethylheptanol; 2-methyl-2-heptanol; 2-methyl-2-octanol; (E)-2-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
of aliphatic aldehydes and their acetals, such as Hexanal; heptanal; octanal; nonanal; decanal; undecanal; dodecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6, 10-trimethyl-9-undecenal; 2,6, 10-trimethyl-5,9-undecadienal; heptanaldiethyl acetal; 1, 1-dimethoxy-2,2,5-trimethyl-4-hexene; citronellyloxyacetaldehyde; 1-(1-methoxy-propoxy)-(E/Z)-3-hexene;
of aliphatic ketones and their oximes such as 2-heptanone; 2-octanone; 3-octanone; 2-nonanone; 5-methyl-3-heptanone; 5-methyl-3-heptanone oxime; 2,4,4,7-tetramethyl-6-octen-3-oη; 6-methyl-5-hepten-2-one;
of aliphatic sulfur-containing compounds such as 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthen-8-thiol;
of aliphatic nitriles such as 2-nonenoic acid nitrile; 2-undecenoic acid nitrile; 2-tridecenoic acid nitrile; 3, 12-tridecadienoic acid nitrile; 3,7-dimethyl-2,6-octadienoic acid nitrile; 3,7-dimethyl-6-octenoic acid nitrile;
esters of aliphatic carboxylic acids, e.g. (E)- and (Z)-3-hexenyl formate; ethyl acetoacetate; isoamyl acetate; 3,5,5-trimethylhexyl acetate; 3-methyl-2-butenyl acetate; (E)-2-hexenyl acetate; (E)- and (Z)-3-hexenyl acetate; Octyl acetate; 3-octyl acetate; 1-octene-3-yl acetate; ethyl butyrate; butyl butyrate; isoamyl butyrate; hexyl butyrate; (E)- and (Z)-3-hexenyl isobutyrate; Hexyl crotonate; ethyl isovalerate; ethyl 2-methylpentanoate; ethyl hexanoate; allyl hexanoate; ethyl heptanoate; allyl heptanoate; ethyl octanoate; Ethyl (E,Z)-2,4-decadienoate; methyl 2-octinate; methyl 2-noninate; allyl 2-isoamyloxyacetate; Methy 1-3,7-dimethyl-2,6-octadiene-oate;4-methyl-2-pentyl crotonate;
of acyclic terpene alcohols such as. Geraniol; nerol; lavadulol; nerolidol; farnesol; tetrahydrolinalool; tetrahydrogeraniol; 2,2-Dimethyl-3-(3-methylphenyl)propan-1-ol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-Dimethyloct-6-en-1-ol; (2E)-3,7-Dimethylocta-2,6-dien-1-ol; 3,7-dimethyl-1,5,7-octatrien-3-ol 2,6-dimethyl-2,5J-octatrien-1-ol; and their formates, acetates, propionates, isobutyrates, butyrates, isovalerianates, pentanoates, hexanoates, crotonates, tiglinates and 3-methyl-2-butenoates;
of the acyclic terpene aldehydes and ketones such as citronellal; 7-methoxy-3,7-dimethyloctanal; 2,6,10-trimethyl-9-undecenal; geranylacetone; and the dimethyl and diethylacetals of geranial, neral,
of cyclic terpene alcohols such as. menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiaol; and their formates, acetates, propionates, isobutyrates, butyrates, isovalerianates, pentanoates, hexanoates, crotonates, tiglinates and 3-methyl-2-butenoates;
of cyclic terpene aldehydes and ketones such as Menthone; isomenthone; 8-mercaptomenthan-3-oη; carvone; camphor; fenchone; alpha-lonone; beta-lonone; alpha-n-methylionone; beta-n-methylionone; alpha-Isomethylionone; beta-Isomethyl-ionone; alpha-irone; beta-damascenone; 1-(2,4,4-trimethyl-2-cyclohexen-1-yl)-2-buten-1-one; 1,3,4,6,7,8a-Hexahydro-1, 1,5,5-tetramethyl-2H-2,4a-methano-naphthalen-8(5H)-on;2-Methyl-4-(2,6,6-trimethyl-1-cyclohexen-1-yl)-2-butenal; Nootkatone; dihydronootkatone; 4,6,8-megastigmatriene-3-oη; alpha-sinensal; beta-sinensal; acetylated cedarwood oil (methylcedrylketone);
of cyclic alcohols such as 4-tert-butylcyclohexanol; 3,3,5-trimethylcyclohexanol; 3-Isocamphylcyclohexanol; 2,6,9-trimethyl-Z2,Z5,E9-cyclododecatrien-1-ol; 2-Isobutyl-4-m ethyltetrahyd ro-2H-pyran-4-ol; 4-cyclohexylbutan-2-ol;
of cycloaliphatic alcohols such as. alpha,3,3-trimethylcyclohexylmethanol; 1-(4-Isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-Methyl-5-(2,2,3-trimethyl-3-cyclopent-1-yl)-4-penten-2-ol; 3,3-Dimethyl-5-(2,2,3-trimethyl-3-cyclopent-1-yl)-4-penten-2-ol; 1-(2,2,6-Trimethylcyclohexyl)pentan-3-ol; 1-(2,2,6-Trimethylcyclohexyl)hexan-3-ol;
cyclic and cycloaliphatic ethers, e.g. Cineol; cedryl methyl ether; cyclododecyl methyl ether;1,1-dimethoxycyclododecane; (ethoxymethoxy)cyclo-dodecane; alpha-cedrene epoxide; 3a,6,6,9a-tetramethyldodecahydro-naphtho[2, 1-b]furan; 3a-ethyl-6,6,9a-trimethyldodecahydronaphtho[2, 1 b]furan; 1,5,9-tri-methyl-13-oxabicyclo[10.1.0]trideca-4,8-diene; rose oxide; 2-(2,4-dimethyl-3-cyclohexen-1-yl)-5-methyl-5-(1-methyl propyl)-1,3-dioxane;
of cyclic and macrocyclic ketones such as 4-tert.-butylcyclohexanone; 2,2,5-trimethyl-5-pentylcyclopentanone; 2-heptylcyclopentanone; 2-pentyl-cyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopenta-decanone; 4-(1-ethoxyvinyl)-3, 3,5,5-tetramethylcyclohexanone; 4-tert.-pentylcyclo-hexanone; 5-cyclohexadecen-1-one; 6,7-dihydro-1, 1,2,3,3-pentamethyl-4(5H)-indanone; 8-cyclohexadecen-1-one; 9-cycloheptadecen-1-oη; cyclopentadecanone; cyclohexadecanone;
of cycloaliphatic aldehydes such as 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
of cycloaliphatic ketones, e.g. 1-(3,3-Dimethylcyclohexyl)-4-penten-1-on; 2,2-Dimethyl-1-(2,4-dimethyl-3-cyclohexen-1-yl)-1-propanon; 1-(5,5-Dimethyl-1-cyclohexen-1-yl)-4-penten-1-on; 2, 3,8,8-tetramethyl-1,2,3,4,5,6,7, 8-octahydro-2-naphthalenyl methyl ketone; methyl 2,6, 10-trimethyl-2,5,9-cyclododecatrienyl ketone; tert.-butyl-(2,4-dimethyl-3-cyclohexen-1-yl)ketone;
of ester of cyclic alcohols such as 2-tert-butylcyclohexyl acetate; 4-tert-butylcyclohexyl acetate; 2-tert-pentylcyclohexyl acetate; 4-tert-pentylcyclohexyl acetate; 3,3, 5-tri methylcyclohexyl acetate; decahydro-2-naphthyl acetate; 2-cyclo-pentylcyclopentyl crotonate; 3-pentyl tetrahydro-2H-pyran-4-yl acetate; decahydro-2,5,5,8a-tetramethyl-2-naphthyl acetate; 4,7-methano-3a,4,5,6,7,7a-hexa-hydro-5, resp. 6-indenyl acetate; 4,7-methano-3a,4,5,6,7,7a-hexahydro-5, or 6-indenyl propionate; 4,7-methano-3a,4,5,6,7,7a-hexahydro-5, or 6-indenyl isobutyrate; 4,7-methanooctahydro-5, or 6-indenyl acetate;
of esters of cycloaliphatic alcohols such as 1-cyclohexylethyl crotonate;
of esters of cycloaliphatic carboxylic acids, e.g. Allyl 3-cyclohexyl propionate; allyl cyclohexyloxy acetate; ice and trans-methyl dihydrojasmonate; ice and trans-methyl jasmonate; methyl 2-hexyl-3-oxocyclopentane carboxylate; ethyl 2-ethyl-6,6-dimethyl-2-cyclohexene carboxylate; ethyl 2,3,6,6-tetramethyl-2-cyclohexene carboxylate; ethyl 2-methyl-1,3-dioxolane-2-acetate;
of araliphatic alcohols such as Benzyl alcohol; 2-phenylethanol; 1-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-Isopropylphenyl)ethanol;
of esters of araliphatic alcohols and aliphatic carboxylic acids, e.g. Benzyl acetate; benzyl propionate; benzyl isobutyrate; benzyl isovalerate; 2-phenylethyl acetate; 2-phenylethyl propionate; 2-phenylethyl isobutyrate; 2-phenyl ethyl isovalerate; 1-phenylethyl acetate; alpha-trichloromethyl benzyl acetate; alpha, alpha-dimethylphenyl ethyl acetate; alpha,alpha-dimethylphenyl ethyl butyrate; cinnamyl acetate; 2-phenoxyethyl isobutyrate; 4-methoxybenzyl acetate;
of araliphatic ethers such as. 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetra-hydroindeno[1,2-d]-m-dioxin; 4,4a,5,9b-tetrahydro-2,4-dimethylindeno[1,2-d]-m-dioxin; of aromatic and araliphatic aldehydes such as. e.g., benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-isobutylphenyl)propanal; 3-(4-tert. butyl-phenyl)propanal; cinnamaldehyde; alpha-butylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
of aromatic and araliphatic ketones such as acetophenone; 4-methylacetophenone; 4-methoxyacetophenone; 4-tert.-butyl-2,6-dimethylaceto-phenone; 4-phenyl-2-butanone; 4-(4-hydroxyphenyl)-2-butanone; 1-(2-naphtha-lenyl)ethanone;2-benzofuranylethanone; (3-methyl-2-benzofuranyl)ethanone; benzophenone; 1,1,2,3,6-hexamethyl-5-indanyl methyl ketone; 6-tert. butyl-1,1-di-methyl-4-indanyl methyl ketone; 1-[2,3-dihydro-1,1,2,6-tetramethyl-3-(1-methylethyl)-1 H-5-indenyl]ethanone; 5′,6′,7′,8′-tetrahydro-3′,5′,5′,6′,8′,8′-hexamethyl-2-aceto-naphthone;
of aromatic and araliphatic carboxylic acids and their esters, e.g. Benzoic acid; phenylacetic acid; methyl benzoate; ethyl benzoate; hexyl benzoate; benzyl benzoate; methyl phenyl acetate; ethyl phenyl acetate; geranyl phenyl acetate; phenyl ethyl phenyl acetate; methyl cinnamate; ethyl cinnamate; benzyl cinnamate; phenyl ethyl cinnamate; cinnamyl cinnamate; allyl phenoxy acetate; methyl salicylate; hexyl salicylate; cyclohexyl salicylate; cis-3-hexenyl salicylate; benzyl salicylate; phenyl ethyl salicylate; methyl 2,4-dihydroxy-3,6-dimethylbenzoate; ethyl 3-phenyl glycidate; ethyl 3-methyl-3-phenyl glycidate;
of heterocyclic compounds such as 2,5-dimethyl-4-hydroxy-2H-furan-3-one; 2-ethyl-4-hydroxy-5-methyl-2H-f u ran-3-oη; 3-hydroxy-2-methyl-4H-pyran-4-one; 2-ethyl-3-hydroxy-4H-pyran-4-one;
of lactones such as. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide;4-methyl-1,4-decanolide; 1,15-pentadecanolide; 1,16-hexadecanolide; 9-hexadecene-1,16-olide; 10-oxa-1,16-hexadecanolide; 1 1-oxa-1,16-hexadecanolide; 12-oxa-1,16-hexadecanolide; ethylene-1,12-dodecanedioate; ethylene-1,13-tridecanedioate; 2,3-dihydrocoumarin; octahydrocoumarin.

In a preferred embodiment according to the present invention, the at least one other fragrance is selected from the group consisting of 4-(4-methoxyphenyl)butan-2-one, 3-Methylbutyl acetate, Hexyl acetate, Benzaldehyde, 3,7-Dimethylocta-1,6-dien-3-ol, 5-heptyloxolan-2-one, 1,3,3-Trimethyl-2-oxabicyclo[2.2.2]octane, 2-(4-methyl-1-cyclohex-3-enyl)propan-2-yl acetate, (2E)-3-Phenylprop-2-enal, 2-Methoxy-4-(prop-2-en-1-yl)phenol, (Z)-dec-6-enal, 3-(1-ethoxyethoxy)-3,7-dimethylocta-1,6-diene, Elettaria cardamomum I. seed oil, Pimenta acris kostel leaf oil, Triethyl 2-hydroxypropane-1,2,3-tricarboxylate, 3-Ethoxy-4-hydroxybenzaldehyde, Phenylmethanol, Cinnamomum verum bark oil, Piper nigrum I. fruit oil black, Benzyl (2E)-3-phenylprop-2-enoate, 3-phenylpropan-1-ol, Syzygium aromaticum I. leaf oil, Myristica fragrans houtt. fruit oil, 5-pentyloxolan-2-one, (2E)-3,7-dimethylocta-2,6-dien-1-ol, Ethyl acetate, 4-Hydroxy-3-methoxybenzaldehyde, (E)-4-(2,6,6-trimethyl-1-cyclohex-2-enyl)but-3-en-2-one, 4-Oxa-2,6-heptandiol and 4-Oxa-1,6-heptandiol, Styrax tonkinensis craib ex hartwiss resin, 2,4-dimethylcyclohex-3-ene-1-carbaldehyde, Benzyl acetate, (2-tert-butylcyclohexyl) acetate, ethyl 2-(2-methyl-1,3-dioxolan-2-yl)acetate.

The fragrance composition according to the invention comprises or consists of linolal and at least one further compound or composition, wherein linolal is contained in a sensory effective amount sufficient to impart, modify and/or enhance fruity and/or spicy notes of an existing fragrance

Preferably, the fragrance composition according to the invention comprises or consists of linolal and two or more further fragrances. More preferably, the fragrance composition according to the invention comprises or consists of linolal and three or more further fragrances. Most preferably, the fragrance composition according to the invention comprises or consists of linolal and four or more further fragrances.

Perfume oil compositions (=fragrance mixtures, compositions) containing linolal are advantageously used for perfuming in liquid form, undiluted or diluted with a solvent. Suitable solvents for this are e.g. Ethanol, isopropanol, diethylene glycol monoethyl ether, glycerin, propylene glycol, 1, 2-butylene glycol, dipropylene glycol, diethyl phthalate, triethyl citrate, isopropyimyristate etc.

Furthermore, fragrance compositions according to the invention can be adsorbed on a carrier, which ensures both a fine distribution of the fragrances in the product and a controlled release during use. Such carriers can be porous inorganic materials such as light sulfate, silica gels, zeolites, plasters, clays, clay granules, aerated concrete, etc. or organic materials such as wood, cellulose-based substances, sugar, dextrins (e.g. maltodextrin) or plastics such as PVC, polyvinyl acetates or polyurethanes. The resulting combination of compositions according to the invention and carrier substance is also to be understood as a fragrance composition according to the invention.

Fragrance compositions according to the invention can also be microencapsulated, spray-dried, as inclusion complexes or as extrusion products and added in this form to a product to be perfumed, for example.

If necessary, the properties of the compositions modified in this way can be further optimized by so-called “coating” with suitable materials with a view to a more targeted release of fragrances, for which purpose waxy plastics such as polyvinyl alcohol are preferably used. The resulting products in turn represent articles according to the invention.

Fragrance compositions according to the invention can advantageously be used in concentrated form, in solutions or in the modified form described above for the production of perfumed articles according to the invention, such as perfume extracts, eau de perfumes, eau de toilettes, aftershave, eau de colognes, pre-shave products, splash colognes and perfumed refreshing towels and the perfuming of acidic, alkaline and neutral cleaning agents such as floor cleaners, window glass cleaners, dishwashing detergents, Bathroom and sanitary cleaners, scouring milk, solid and liquid toilet cleaners, powder and foam carpet cleaners, textile fresheners, ironing aids, liquid detergents, powder detergents, laundry pretreatment agents such as bleaches, soaking agents and stain removers, fabric softeners, laundry soaps, washing tablets, and disinfectants Air fresheners in liquid, gel-like or applied form on a solid carrier, aerosol sprays, waxes and polishes such as furniture polishes, floor waxes, shoe creams and personal care products such as solid and liquid soaps, shower gels, shampoos, shaving soaps, shaving foams s, bath oils, cosmetic emulsions of the oil-in-water, of the water-in-oil and of the water-in-oil-in-water type such as, for example, skin creams and lotions, face creams and lotions, sun protection creams and lotions, after-sun creams and lotions, hand creams and lotions, foot creams and lotions, depilatory creams and lotions, after-shave creams and lotions, tanning creams and lotions, hair care products such as hair sprays, hair gels, setting hair lotions, Hair conditioners, permanent and semi-permanent hair dyes, hair shaping agents such as cold waves and hair straighteners, hair lotions, hair creams and lotions, deodorants and antiperspirants such as underarm sprays, roll-ons, deodorant sticks, deodorant creams, products in decorative cosmetics such as eye shadow, nail varnish, Lipsticks, mascara and candles, lamp oils, incense sticks, insecticides, repellants and fuels.

Of course, fragrance compositions according to the invention can also be included in cosmetic compositions or household compositions.

Another aspect of the invention relates to a method for modifying and/or enhancing an existing fragrance to impart, modify and/or enhance fruity and/or spicy notes of an existing fragrance, comprising or consisting of the following steps:
a. Providing linolal,
b. Providing at least one further fragrance
c. adding linolal to the at least one further fragrance in a sensory effective amount sufficient to impart, modify and/or enhance fruity and/or spicy notes of an existing fragrance.

A further aspect of the present invention relates to a perfumed product comprising or consisting of linolal and at least one further fragrance compound, wherein linolal is contained in a sensory effective amount sufficient to impart, modify and/or enhance fruity and/or spicy notes of an existing fragrance.

In a preferred embodiment the perfumed product is selected from cosmetic, hygiene and/or household articles. Further preferred embodiments listed above also apply to the perfumed product of the present invention.

Preferably, the perfumed household article is selected from the group consisting of detergents and cleaning agents, hygiene or care products, preferably in the field of body and hair care, cosmetics and household, preferably from the group consisting of perfume extracts, Eau de perfumes, eau de toilets, aftershave lotions, eau de colognes, pre-shave products, splash colognes, perfumed refreshing tissues, acidic, alkaline or neutral cleaning agents, textile fresheners, ironing aids, liquid detergents, powder detergents, laundry pretreatment agents, fabric softeners, Wash tablets, disinfectants, surface disinfectants, air fresheners, aerosol sprays, waxes and polishes, personal care products, hand creams and lotions, foot creams and lotions, depilatory creams and lotions, after shave creams and lotions, tanning creams and lotions, hair care products, hair care products, Products of the decorative cosmetics, candles, lamp oils, incense sticks, insecticides, repellants and fuels. Most preferably, the perfumed product is selected from alcoholic perfume, a personal hygiene product or a cleaning or care product for use in the home.

The additives, auxiliaries and/or active substances described above are preferably not odoriferous substances. These can be, for example, preservatives, antibacterial agents, chelating agents, cleaning agents, emulsifiers, fats, etc. and in principle all substances that are used as additives, auxiliaries and/or active ingredients in cosmetics, especially in fragrance compositions, as well as in household compositions.

Further preferred embodiments listed above also apply to the perfumed product of the present invention.

In the following, the invention is further characterized on the basis of the examples.

### EXAMPLES

### Example 1-13: Impact of Trace Amounts of Linolal on Fragrance Compositions

In order to explore the effect of linolal on boosting or modifying scent profiles, known fragrance compounds or compositions have been mixed with working amounts of linolal. Changes of the resulting scent in comparison to compositions without linolal have been reported in Table 1. Amount of linolal in % b.w. of the total composition.

TABLE 1
Addition of trace amounts of linolal enhances
and modifies fragrance profiles

| Nr | Name | linolal | Fragrance |
|---|---|---|---|
| 1 | FRAMBINON ® | 0.05% | More watery. push the |
| | METHYL ETHER | | watermelon effect |
| | | | More raspberry |
| | | | More ozonic |
| 2 | Isoamyl Acetate | 0.05% | Less acetate |
| | | | More banana |
| | | | More natural |
| 3 | Hexyl acetate | 0.05% | More green hexenal like |
| | | | More fruity |
| | | | More pear juicy |
| 4 | Benzaldehyde DD | 0.1% | Push the cinnamate effect |
| | | | More spicy |
| | | | Less cherry. more almond |
| 5 | LINALOOL | 0.1% | More coriander |
| | | | More basil |
| | | | Gives a bergamot peel natural effect |
| 6 | Aldehyde C14 | 0.05% | More lactonic |
| | so-called | | More apricot. fatty |
| | | | More natural peach/apricot note |
| 7 | EUCALYPTOL | 0.1% | More eucalyptus natural |
| | NAT. | | More powerful |
| | | | More complex |
| 8 | TERPINYL | 0.1% | More linalyl acetate |
| | ACETATE | | Less animalic. more peppery |
| | | | More powerful |
| 9 | CINNAMIC | 0.1% | More cinnamon bark natural |
| | ALDEHYDE | | Gives cassia effect |
| | | | More powerful |
| 10 | EUGENOL NAT. | 0.05% | More bay oil |
| | | | More metallic |
| | | | Less carnation |
| 11 | Decenal-6.4 | 0.3% | More mandarin peel |
| | | | Less fatty |
| | | | More juicy citrus |
| 12 | Belanis | 0.3% | More powerful |
| | | | More caraway |
| | | | More anise natural |
| 13 | Elintal forte | 0.3% | Less algae |
| | | | More cardamom |
| | | | More spicy |

These data illustrate the strong impact of linolal on fragrance compositions in order to modify and enhance already present notes.

### Example 14: Fragrance Composition Containing Small Amounts of Linolal

TABLE 2
Fragrance mixture

| Material Description | NOW |
|---|---|
| ALDEHYDE C18 SO-CALLED | 12.000 |
| GERANIOL SUPER | 6.000 |
| TRIETHYL CITRATE | 0.000 |
| ISOAMYL ACETATE | 1.000 |
| ETHYL ACETATE | 3.000 |
| LINALOOL | 30.000 |
| VANILLIN | 4.000 |
| IONONE ALPHA | 4.000 |
| DIPROPYLENE GLYCOL | 840.000 |
| BENZOIN SIAM RESIN 50% IN BB | 4.000 |
| LIGUSTRAL 10% DPG | 1.000 |
| linolal 10% DPG | 5.000 |
| BENZALDEHYDE DD 10% DPG | 1.000 |
| ALDEHYDE C14 SO-CALLED | 80.000 |
| BENZYL ACETATE | 2.000 |
| AGRUMEX HC | 5.000 |
| JASMAPRUNAT | 2.000 |
| | 1,000.000 |

According to an expert panel, linolal raw material gives to this fruity accord much more juiciness and enhance the natural peachy notes by its sparkling effect. The overall fragrance is boosted and display a much nicer fruity profile.

### Example 15: Fragrance Composition Containing Small Amounts of Linolal

TABLE 3
Fragrance mixture

| Material Description | NOW |
|---|---|
| CARDAMOM OIL | 10.000 |
| BAY OIL MADA | 10.000 |
| TRIETHYL CITRATE | 653.000 |
| ETHYL VANILLIN | 30.000 |
| BENZYL ALCOHOL DD | 30.000 |
| CINNAMON BARK OIL MADAG. | 100.000 |
| SAFROL FREE | |
| PEPPER BLACK FRESH OIL MADA | 20.000 |
| LINOLAL | 2.000 |
| BENZYL CINNAMATE | 20.000 |
| CINNAMIC ALDEHYDE | 50.000 |
| PHENYLPROPYL ALCOHOL | 10.000 |
| CLOVE LEAF OIL DECOL. | 50.000 |
| NUTMEG OIL LOW SAFROLE | 15.000 |
| | 1,000.000 |

According to an expert panel, the sparkling cinnamon note is boosted and more impactful thanks to the metallic note of linolal. It overall boosts all the spiciness of the fragrance and can be used as traces to give power to spicy top notes.

The presented examples clearly show the impact of trace amounts of linolal on modifying and boosting particularly spicy and fruity notes in fragrance compositions.

1. Use of linolal and at least one further compound and/or composition within a fragrance composition, wherein linolal is used in a sensory effective amount sufficient to impart, modify and/or enhance fruity and/or spicy notes of an existing fragrance.

2. Use according to claim 1, wherein the fruity notes are selected from the group consisting of watermelon, raspberry, banana, pear, apricot, peach, mandarin peel, and citrus.

3. Use according to claim 1 and/or 2, wherein the spicy notes are selected from the group consisting of almond, coriander, basil, bergamot peel, eucalyptus, pepper, cinnamon, cassia, bay oil, caraway, anise, and cardamom.

4. Use according to at least one of claims 1 to 3, wherein the at least one further compound is selected from the group consisting of 4-(4-methoxyphenyl)butan-2-one, 3-Methylbutyl acetate, Hexyl acetate, Benzaldehyde, 3,7-Dimethylocta-1,6-dien-3-ol, 5-heptyloxolan-2-one, 1,3,3-Trimethyl-2-oxabicyclo[2.2.2]octane, 2-(4-methyl-1-cyclohex-3-enyl)propan-2-yl acetate, (2E)-3-Phenylprop-2-enal, 2-Methoxy-4-(prop-2-en-1-yl)phenol, (Z)-dec-6-enal, 3-(1-ethoxyethoxy)-3,7-dimethylocta-1,6-diene, Elettaria cardamomum I. seed oil, Pimenta acris kostel leaf oil, Triethyl 2-hydroxypropane-1,2,3-tricarboxylate, 3-Ethoxy-4-hydroxybenzaldehyde, Phenylmethanol, Cinnamomum verum bark oil, Piper nigrum I. fruit oil black, Benzyl (2E)-3-phenylprop-2-enoate, 3-phenylpropan-1-ol, Syzygium aromaticum I. leaf oil, Myristica fragrans houtt. fruit oil, 5-pentyloxolan-2-one, (2E)-3,7-dimethylocta-2,6-dien-1-ol, Ethyl acetate, 4-Hydroxy-3-methoxybenzaldehyde, (E)-4-(2,6,6-trimethyl-1-cyclohex-2-enyl)but-3-en-2-one, 4-Oxa-2,6-heptandiol and 4-Oxa-1,6-heptandiol, Styrax tonkinensis craib ex hartwiss resin, 2,4-dimethylcyclohex-3-ene-1-carbaldehyde, Benzyl acetate, (2-tert-butylcyclohexyl) acetate, ethyl 2-(2-methyl-1,3-dioxolan-2-yl)acetate.

5. Use according to at least one of claims 1 to 4, wherein the amount of linolal is in the range of 0.001 to 5% by weight, based on the total weight of the fragrance composition.

6. Use according to at least one of claims 1 to 4, wherein the amount of linolal is in the range of 0.05 to 1% by weight, based on the total weight of the fragrance composition

7. Use according to at least one of claims 1 to 4, wherein the weight ratio of linolal to the total amount of other fragrances is in the range of 1:100000 to 1:20.

8. Use according at least one of claims 1 to 4, wherein the weight ratio of linolal to the total amount of other fragrances is in the range of 1:20000 to 1:1000.

9. Fragrance composition comprising or consisting of linolal and at least one further compound, wherein linolal is contained in a sensory effective amount sufficient to impart, modify and/or enhance fruity and/or spicy notes of an existing fragrance.

10. Fragrance composition according to claim 9, wherein the amount of linolal is in the range of 0.001 to 5% by weight, based on the total weight of the fragrance composition.

11. Fragrance composition according to claim 9, wherein the weight ratio of linolal to the total amount of other fragrances is in the range of 1:100000 to 1:20.

12. Method for modifying and/or enhancing an existing fragrance to impart, modify and/or enhance fruity and/or spicy notes of an existing fragrance, comprising or consisting of the following steps:
a. Providing linolal,
b. Providing at least one further fragrance
c. adding linolal to the at least one further fragrance in a sensory effective amount sufficient to impart, modify and/or enhance fruity and/or spicy notes of an existing fragrance.

13. Method according to claim 12, wherein the amount of linolal is in the range of 0.001 to 5% by weight, based on the total weight of the fragrance composition.

14. Method according to claim 12, wherein the weight ratio of linolal to the total amount of other fragrances is in the range of 1:100000 to 1:20.

15. Perfumed product comprising or consisting of linolal and at least one further fragrance compound wherein linolal is contained in a sensory effective amount sufficient to impart, modify and/or enhance fruity and/or spicy notes of an existing fragrance.
